(19) United States
(12) Patent Application Publication
(10) Pub. No.: US 2025/0004556 A1
(43) Pub. Date: Jan. 2, 2025

(54) COMMUNICATION DEVICE, COMMUNICATION METHOD, AND COMPUTER-READABLE MEDIUM

(71) Applicant: Yokogawa Electric Corporation, Tokyo (JP)

(72) Inventors: Kiyomi MURAOKA, Tokyo (JP); Hirotaka YAMASHITA, Tokyo (JP); Takashi TSUBOTA, Tokyo (JP); Motohiko ISHIDA, Tokyo (JP)

(21) Appl. No.: 18/744,677

(22) Filed: Jun. 16, 2024

(30) Foreign Application Priority Data
Jun. 27, 2023 (JP) 2023-104702

(51) Int. Cl. G06F 3/01; A61B 5/374; B25J 11/00; B25J 13/00; B25J 13/08; G10L 15/24

(52) U.S. Cl. CPC G06F 3/015; A61B 5/374; B25J 11/0005; B25J 13/003; B25J 13/087; G10L 15/24

(57) ABSTRACT

There is provided a communication device in which a reaction changes according to a state of an evaluation target person. The communication device includes an information acquisition unit which acquires brainwave information of the evaluation target person; a reaction determination unit which determines a first reaction of the communication device, based on the brainwave information; and a control unit which controls the communication device according to the first reaction determined by the reaction determination unit. The reaction determination unit may determine a content of an utterance of the communication device, based on the brainwave information. The control unit may control the communication device according to the content of the utterance determined by the reaction determination unit.

The contents of the following patent applications are incorporated herein by reference: NO. 2023-104702 filed in JP on Jun. 27, 2023

### BACKGROUND

### 1. Technical Field

The present invention relates to a communication device, a communication method, and a computer-readable medium.

### 2. Related Art

Patent Document 1 discloses that “ . . . increase satisfaction with a conversation with a conversational agent device (Abstract)”. Patent Document 2 discloses that “ . . . estimates, with high accuracy, a target image that is being recalled by a person (Abstract)”.

### PRIOR ART DOCUMENT

### Patent Document

Patent Document 1: Japanese Patent Application Publication No. 2021-114004
Patent Document 2: International Publication No. WO2019/235458

### BRIEF DESCRIPTION OF THE DRAWINGS

FIG. 1 is a diagram showing an example of a communication between an evaluation target person 120 and a communication device 100.

FIG. 2 is a diagram showing an example of the communication between the evaluation target person 120 and the communication device 100.

FIG. 3 is a block diagram showing an example of the communication device 100 according to an embodiment of the present invention.

FIG. 4 is a diagram showing an example of an electroencephalograph 14.

FIG. 5 is a diagram showing an example of state information Is.

FIG. 6 is a diagram showing an example of a state inference model 42.

FIG. 7 is a flowchart showing an example of a communication method according to an embodiment of the present invention.

FIG. 8 is a diagram showing an example of a computer 2200 in which the communication device 100 according to an embodiment of the present invention may be implemented in whole or in part.

### DESCRIPTION OF EXEMPLARY EMBODIMENTS

Hereinafter, the present invention will be described through embodiments of the invention, but the following embodiments do not limit the invention according to claims. In addition, not all combinations of features described in the embodiment are essential to the solution of the invention.

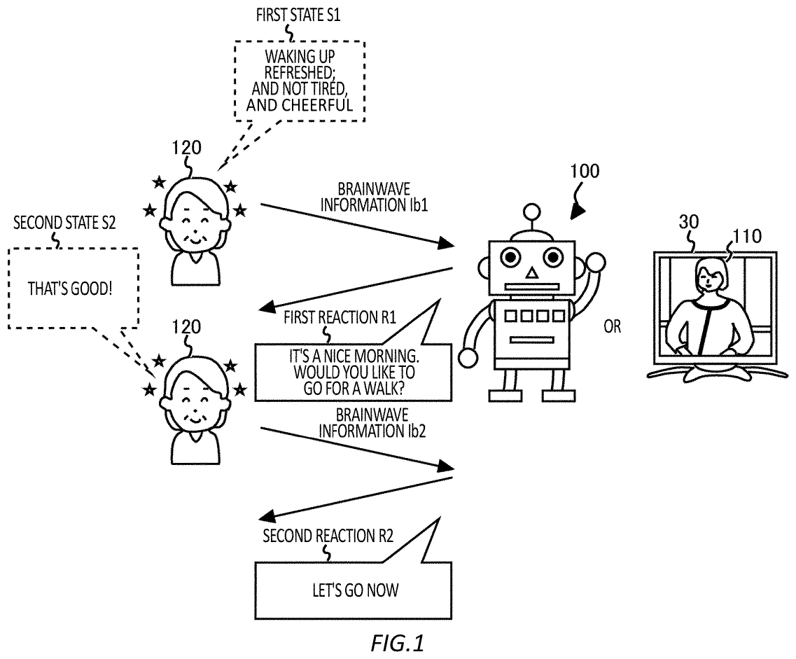

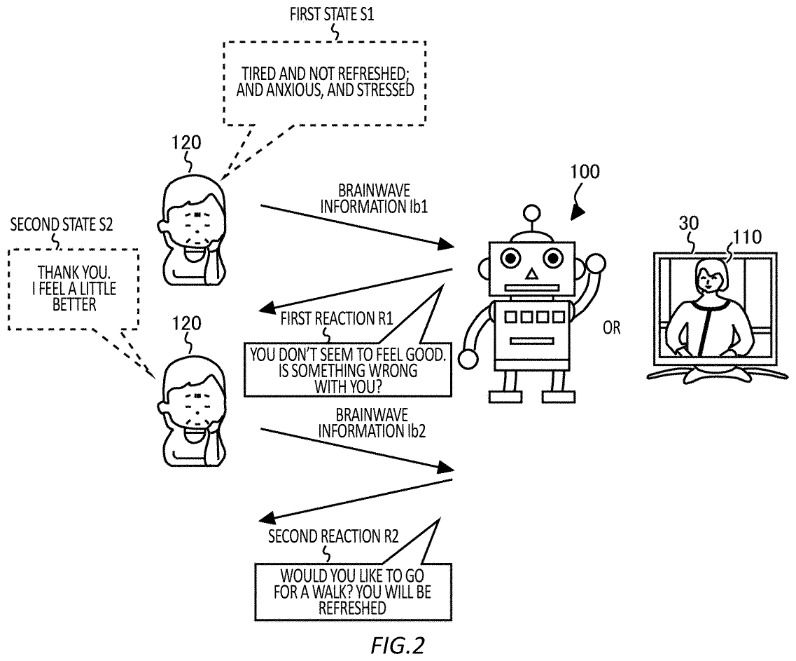

FIG. 1 and FIG. 2 are diagrams showing examples of a communication between an evaluation target person 120 and a communication device 100. In the present example, the evaluation target person 120 is a human. The communication device 100 may be a device simulating a human or an animal. The communication device 100 may be a tangible object such as a robot, or may be a living body 110 on a computer. The living body 110 may be a virtual person or a virtual animal. The living body 110 may be presented to a reaction presentation unit 30 (described below).

A state of the evaluation target person 120 is set as a state S. The state S may be a potential state of the evaluation target person 120. The potential state of the evaluation target person 120 is a psychological state of the evaluation target person that the evaluation target person 120 is not aware of. FIG. 1 and FIG. 2 are examples after the evaluation target person 120 wakes up. The state S of the evaluation target person 120 before a first reaction R1 (described below) is received, is set as a first state S1. The state S of the evaluation target person 120 after the first reaction R1 (described below) is received and before a second reaction R2 (described below) is received, is set as a second state S2. The first state S1 and the second state S2 of the evaluation target person 120 are shown in broken line parts of FIG. 1 and FIG. 2. In the example of FIG. 1, before the first reaction R1 (described below) is received, the evaluation target person 120 feels “waking up refreshed; and not tired, and cheerful”. In the example of FIG. 2, before the first reaction R1 (described below) is received, the evaluation target person 120 feels “tired and not refreshed; and anxious, and stressed”.

A reaction of the communication device 100 changes according to the state S of the evaluation target person 120. The reaction of the communication device 100 refers to an utterance, an action, a facial expression, or the like of the communication device 100. Brainwave information Ib1 and brainwave information Ib2 in FIG. 1 and FIG. 2 will be described below.

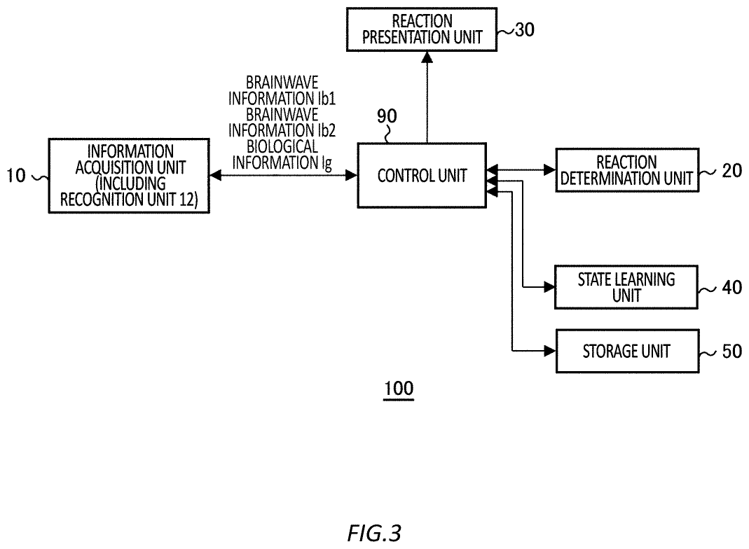

FIG. 3 is a block diagram showing an example of the communication device 100 according to an embodiment of the present invention. The communication device 100 includes an information acquisition unit 10, a reaction determination unit 20, and a control unit 90. The communication device 100 may include the reaction presentation unit 30, a state learning unit 40, and a storage unit 50. The information acquisition unit 10 may include a recognition unit 12. The reaction presentation unit 30 is, for example, a display, a monitor, or the like.

A part or all of the communication device 100 may be realized by the computer. The control unit 90 may be a Central Processing Unit (CPU) of this computer. When the communication device 100 is realized by the computer, a program for causing the computer to function as the communication device 100 may be installed on the computer, or a program may be installed for executing a communication method described below.

Brainwave information of the evaluation target person 120 is set as brainwave information Ib. The information acquisition unit 10 acquires the brainwave information Ib of the evaluation target person 120. The brainwave information Ib may be information that reproduces at least a part of a temporal waveform of a brainwave of the evaluation target person 120. The brainwave information Ib may include data obtained by sampling the temporal waveform of the brainwave, may include data indicating magnitudes of frequency components of brainwaves at one or more frequencies, or may include another piece of data. For example, the brainwave information Ib includes data indicating a magnitude of at least one component of an alpha wave, a beta wave, a theta wave, a delta wave, or a gamma wave.

The alpha wave may be further classified by a frequency band into a high alpha wave, a medium alpha wave, and a low alpha wave. The beta wave may be classified into a high beta wave and a low beta wave. The brainwave information Ib may include data indicating the magnitude of at least one of the high alpha wave, the medium alpha wave, or the low alpha wave. The brainwave information Ib may include data indicating the magnitude of at least one of the high beta wave or the low beta wave.

The brainwave information Ib may include information of the temporal waveforms of one or more brainwaves measured at one or more positions on a head part including a head and a face of the evaluation target person 120. For example, the brainwave information Ib may be acquired by measuring the temporal waveforms of potentials of electrodes arranged at equal intervals near a scalp of the evaluation target person 120, as in the international 10-20 system, or may be acquired by another method. The intervals of a plurality of electrodes that are arranged on the scalp may not be equal. The electrode may be provided on a wearable device which is worn on the head of the evaluation target person 120, such as a headgear, a headphone, earphones, glasses, or the like. The brainwave information Ib may be information of an electrical signal at an electrode implanted in a body of the evaluation target person 120, which is acquired by a wireless communication. In the examples of FIG. 1 and FIG. 2, the brainwave information Ib is wirelessly transmitted to the control unit 90.

The reaction determination unit 20 determines the first reaction of the communication device 100, based on the brainwave information Ib1. The first reaction is set as the first reaction R1. The brainwave information Ib1 is the brainwave information Ib before the first reaction R1. For example, the reaction determination unit 20 determines the first reaction R1 or the second reaction R2 (described below), based on the magnitude of a specific frequency component of the brainwave of the evaluation target person 120. The reaction determination unit 20 may determine the first reaction R1, or may determine the second reaction R2 (described below), based on the magnitudes of one or more components among the alpha wave, the beta wave, the theta wave, the delta wave, and the gamma wave.

At a certain timing, a sum of amplitudes of the alpha wave (8 Hz or more and less than 14Hz), the beta wave (14 Hz or more and less than 26 Hz), the theta wave (4 Hz or more and less than 8 Hz), the gamma wave (26 Hz or more and less than 40 Hz), and the delta wave (less than 4 Hz) is set as a total amplitude As. As an example, in a case where a ratio of the amplitude of the delta wave (less than 4 Hz) of the evaluation target person 120 to the total amplitude As is greater than a ratio of the amplitude of the alpha wave to the total amplitude As; a ratio of the amplitude of the beta wave to the total amplitude As; a ratio of the amplitude of the theta wave to the total amplitude As; and a ratio of the amplitude of the gamma wave to the total amplitude As, it can be inferred that the evaluation target person 120 is in a state of sleep. As an example, in a case where the ratio of the amplitude of the theta wave of the evaluation target person 120 to the total amplitude As, increases over time, it can be inferred that tiredness and sleepiness of the evaluation target person 120 is increasing.

As an example, in a case where a ratio of a sum of the amplitude of the low alpha wave (8Hz or more and less than 10 Hz) and the amplitude of the medium alpha wave (10 Hz or more and less than 12 Hz) of the evaluation target person 120 to the total amplitude As, increases over time, it can be inferred that a degree of relaxation of the evaluation target person 120 is increasing.

As an example, in a case where a ratio of a sum of the amplitude of the high alpha wave (12 Hz or more and less than 14 Hz) and the amplitude of the low beta wave (14 Hz or more and less than 18 Hz) of the evaluation target person 120 to the total amplitude As, increases over time, it can be inferred that a state in which a balance between the relaxation and a concentration of the evaluation target person 120 is good, is increasing. The state in which the balance between the relaxation and the concentration is good, is a so-called a state of immersion.

The greater a ratio of a sum of the amplitude of the low alpha wave and the amplitude of the medium alpha wave of the evaluation target person 120 to the total amplitude As, is, the higher a probability that the degree of the relaxation of the evaluation target person 120 is high, is. In this case, the reaction determination unit 20 may determine the first reaction R1 or the second reaction R2 (described below) for the evaluation target person 120 who is in a state of relaxation. The greater a ratio of the sum of the amplitude of the high alpha wave and the amplitude of the low beta wave of the evaluation target person 120 to the total amplitude As, is, the higher the probability that a degree of the immersion of the evaluation target person 120 is high, is. In this case, the reaction determination unit 20 may determine the first reaction R1 or the second reaction R2 (described below) for the evaluation target person 120 who is in the state of immersion.

The reaction determination unit 20 may combine the plurality of components among the alpha wave, the beta wave, theta wave, the delta wave, and the gamma wave of the evaluation target person 120, and determine the first reaction R1 or the second reaction R2 (described below). For example, the greater the value obtained by dividing the magnitude of the alpha wave of the evaluation target person 120 by the magnitude of the beta wave, is, the higher the probability that the degree of the relaxation of the evaluation target person 120 is high, is. Therefore, the reaction determination unit 20 may determine the first reaction R1 or the second reaction R2 (described below) for the evaluation target person 120 who is in the state of relaxation.

The first reaction R1 and the second reaction R2 (described below) may be the utterance of the communication device 100, may be the action, or may be the facial expression. In the examples of FIG. 1 and FIG. 2, the reaction determination unit 20 determines a content of the utterance of the communication device 100 based on the brainwave information Ib1, and determines a content of another utterance of the communication device 100 based on the brainwave information Ib2 (described below).

The control unit 90 controls the communication device 100 according to the first reaction R1 determined by the reaction determination unit 20. In the examples of FIG. 1 and FIG. 2, the control unit 90 controls the communication device 100 according to the content of the utterance determined by the reaction determination unit 20. In the examples of FIG. 1 and FIG. 2, the control unit 90 controls the content of the utterance of the communication device 100.

The potential first state S1 of the evaluation target person 120 can be reflected in the brainwave information Ib1. The first reaction R1 is determined based on the brainwave information Ib1. Therefore, the first reaction R1 is likely to be a reaction corresponding to the potential first state S1 of the evaluation target person 120. In the example of FIG. 1, the first state S1 of the evaluation target person 120, which is “waking up refreshed”, can be reflected in the brainwave information Ib1. Therefore, in the example of FIG. 1, the first reaction R1 is an utterance “It's a nice morning. Would you like to go for a walk?”. In the example of FIG. 2, the first state S1 of the evaluation target person 120, which is “tired and not refreshed”, can be reflected in the brainwave information Ib1. Therefore, in the example of FIG. 2, the first reaction R1 is an utterance “You don't seem to feel good. Is something wrong with you?”.

The brainwave information Ib of the evaluation target person 120 after the first reaction R1 is set as the brainwave information Ib2. The brainwave information Ib2 is the brainwave information Ib of the evaluation target person 120 who has received the first reaction R1 of the communication device 100. The information acquisition unit 10 may acquire a change in the brainwave information Ib from before to after the first reaction R1. The change in the brainwave information Ib from before to after the first reaction R1 is a change from the brainwave information Ib1 to the brainwave information Ib2.

The reaction determination unit 20 may determine the second reaction, based on the brainwave information Ib and biological information Ig (described below) after the first reaction R1. The second reaction is set as the second reaction R2. The reaction determination unit 20 may determine the second reaction R2 of the communication device 100, based on the change from the brainwave information Ib1 to the brainwave information Ib2. The reaction determination unit 20 may determine the second reaction R2, based on the change from the brainwave information Ib1 to the brainwave information Ib2, and the biological information Ig (described below). The control unit 90 may control the communication device 100 according to the second reaction R2 determined by the reaction determination unit 20. The reaction determination unit 20 may determine the second reaction R2, based on the first reaction R1, and the change from the brainwave information Ib1 to the brainwave information Ib2.

When a ratio of a sum of the amplitude of the low alpha wave and the amplitude of the medium alpha wave to the total amplitude As in the brainwave information Ib2, is greater than a ratio of a sum of the amplitude of the low alpha wave and the amplitude of the medium alpha wave to the total amplitude As in the brainwave information Ib1, there is a high probability that the degree of the relaxation of the evaluation target person 120 is increasing. In this case, the reaction determination unit 20 may determine the second reaction R2 for the evaluation target person 120 who is in the state of relaxation. When a ratio of a sum of the amplitude of the high alpha wave and the amplitude of the low beta wave to the total amplitude As in the brainwave information Ib2, is greater than a ratio of a sum of the amplitude of the high alpha wave and the amplitude of the low beta wave to the total amplitude As in the brainwave information Ib1, there is a high probability that the degree of the immersion of the evaluation target person 120 is increasing. In this case, the reaction determination unit 20 may determine the second reaction R2 for the evaluation target person 120 who is in the state of immersion.

The potential second state S2 of the evaluation target person 120 who has received the first reaction R1 can be reflected in the brainwave information Ib2. The second reaction R2 is determined based on the brainwave information Ib2. Therefore, the second reaction R2 is likely to be a reaction corresponding to the potential second state S2 of the evaluation target person 120. In the example of FIG. 1, the evaluation target person 120 who has received the first reaction R1 feels, “That's good!”. In the example of FIG. 2, the evaluation target person 120 who has received the first reaction R1 feels, “Thank you. I feel a little better”.

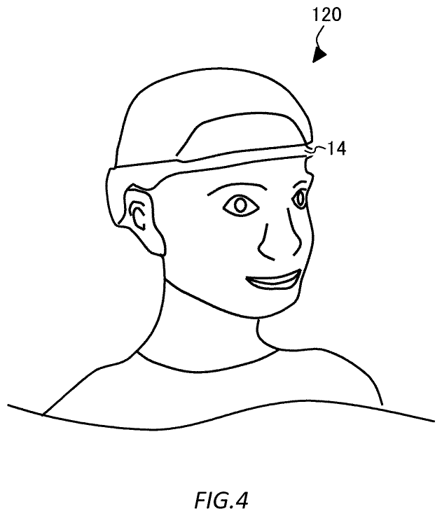

FIG. 4 is a diagram showing an example of an electroencephalograph 14. The electroencephalograph 14 of the present example is of a headgear type. The electroencephalograph 14 may be of an earphone type. In the present example, the evaluation target person 120 may communicate with the communication device 100, in a state in which the electroencephalograph of the headgear type or the earphone type is worn. The electroencephalograph 14 acquires the brainwave information Ib. In the present example, the information acquisition unit 10 (refer to FIG. 3) wirelessly acquires the brainwave information Ib acquired by the electroencephalograph 14.

The reaction presentation unit 30 may be the display, the monitor, or the like installed separately from a housing of the headgear shown in FIG. 4. When the reaction presentation unit 30 is a sound generating device such as a speaker, the reaction presentation unit 30 may or may not be housed in the housing of the headgear shown in FIG. 4. When the reaction presentation unit 30 is the headgear such as a VR (Virtual Reality) headset, the reaction presentation unit 30 may be housed in the housing of the headgear shown in FIG. 4.

The biological information of the evaluation target person 120 is set as the biological information Ig. The biological information Ig may be biological information in a state in which the evaluation target person 120 (refer to FIG. 1 and FIG. 2) is communicating with the communication device 100. The biological information Ig may include at least one of heart rate information, information of an amount of sweat, or body temperature information of the evaluation target person 120. The biological information Ig of the evaluation target person 120 may be acquired by a sensor provided in the wearable device (for example, the electroencephalograph 14 of the headgear type shown in FIG. 4) worn by the evaluation target person 120.

The information acquisition unit 10 (refer to FIG. 3) may further acquire the biological information Ig of the evaluation target person 120. The reaction determination unit 20 (refer to FIG. 3) may determine the first reaction R1, based on the brainwave information Ib and the biological information Ig. The potential state S of the evaluation target person 120 is likely to be reflected in the biological information Ig. For example, when the evaluation target person 120 feels stressed, the evaluation target person 120 is likely to be in a state in which sympathetic nerves are more dominant than parasympathetic nerves. When the sympathetic nerves are more dominant than the parasympathetic nerves, a heart rate fluctuation of the evaluation target person 120 is likely to be small, and an amount of sweat is likely to be great. Therefore, by the first reaction R1 being determined based on the brainwave information Ib and the biological information Ig, the reaction determination unit 20 (refer to FIG. 3) can determine the first reaction R1 that can more appropriately correspond to the potential state S1 of the evaluation target person 120. The information acquisition unit 10 may acquire the biological information Ig before the first reaction R1, or may acquire the biological information Ig after the first reaction R1.

The reaction determination unit 20 may generate state information Is, based on the brainwave information Ib and the biological information Ig. The state information Is is information based on the potential state S of the evaluation target person 120. The reaction determination unit 20 may generate the state information Is, based on the change from the brainwave information Ib1 to the brainwave information Ib2, and the biological information Ig. The reaction determination unit 20 may determine the second reaction R2 based on the state information Is.

The magnitude of a first power spectrum in the heart rate of the evaluation target person 120 is set as an LF, and the magnitude of a second power spectrum is set an HF. A frequency band of the second power spectrum is a band in which a frequency is higher than that in a frequency band of the first power spectrum. The frequency band of the first power spectrum and the frequency band of the second power spectrum may not overlap each other. The frequency band of the first power spectrum is, for example, 0.04 Hz to 0.15 Hz. The frequency band of the second power spectrum is, for example, 0.15 Hz to 0.4 Hz.

A change from a ratio of the amplitudes of the high beta wave (18 Hz or more and less than 26 Hz) and the gamma wave to the total amplitude As in the brainwave information Ib1, to a ratio of the amplitudes of the high beta wave and the gamma wave to the total amplitude As in the brainwave information Ib2, is set as a change C1. The reaction determination unit 20 may generate the state information Is, based on the change C1 and a ratio of the LF to the HF (LF/HF).

As an example, when a ratio of a sum of the amplitude of the high beta wave and the amplitude of the gamma wave of the evaluation target person 120 to the total amplitude As after the first reaction R1, is greater than a ratio of a sum of the amplitude of the high beta wave and the amplitude of the gamma wave of the evaluation target person 120 to the total amplitude As before the first reaction R1, and the ratio of the LF to the HF (LF/HF) after the first reaction R1 is greater than or equal to a threshold value, it can be inferred that a state of irritation, a state of high nervousness, or a state of stress of the evaluation target person 120 is increasing. In this case, the reaction determination unit 20 may determine the second reaction R2 corresponding to the state of irritation, the state of high nervousness, or the state of stress of the evaluation target person 120.

When the ratio of the LF to the HF (LF/HF) is greater than or equal to a threshold value, it may be determined that the evaluation target person 120 is in a state in which the sympathetic nerves are more dominant than the parasympathetic nerves. When the ratio of the LF to the HF (LF/HF) is less than the threshold value, it may be determined that the evaluation target person 120 is in a state in which the parasympathetic nerves are more dominant than the sympathetic nerves. The threshold value may be 2, may be 3, may be 4, or may be 5.

As an example, when a ratio of a sum of the amplitude of the high beta wave and the amplitude of the gamma wave of the evaluation target person 120 to the total amplitude As after the first reaction R1, is greater than a ratio of a sum of the amplitude of the high beta wave and the amplitude of the gamma wave of the evaluation target person 120 to the total amplitude As before the first reaction R1, and the ratio of the LF to the HF (LF/HF) after the first reaction R1 is less than a threshold value, it can be inferred that a state of excitement of the evaluation target person 120 is increasing. In this case, the reaction determination unit 20 may determine the second reaction R2 corresponding to the state of excitement of the evaluation target person 120.

The reaction determination unit 20 may generate the state information Is, based on a magnitude relationship between the ratio of the LF to the HF (LF/HF) after the first reaction R1, and a threshold value of the ratio of the LF to the HF; and the change C1. The threshold value may be determined in advance. When a ratio of a sum of the amplitude of the high beta wave and the amplitude of the gamma wave of the evaluation target person 120 to the total amplitude As after the first reaction R1, is greater than a ratio of a sum of the amplitude of the high beta wave and the amplitude of the gamma wave of the evaluation target person 120 to the total amplitude As before the first reaction R1, and the ratio of the LF to the HF (LF/HF) after the first reaction R1 is greater than or equal to the threshold value, the reaction determination unit 20 may generate the state information Is indicating that a sense of alertness of the evaluation target person 120 is increasing. When a ratio of a sum of the amplitude of the high beta wave and the amplitude of the gamma wave of the evaluation target person 120 to the total amplitude As after the first reaction R1, is greater than a ratio of a sum of the amplitude of the high beta wave and the amplitude of the gamma wave of the evaluation target person 120 to the total amplitude As before the first reaction R1, and the ratio of the LF to the HF (LF/HF) after the first reaction R1 is less than the threshold value, the reaction determination unit 20 may generate the state information Is indicating that a sense of excitement of the evaluation target person 120 is increasing.

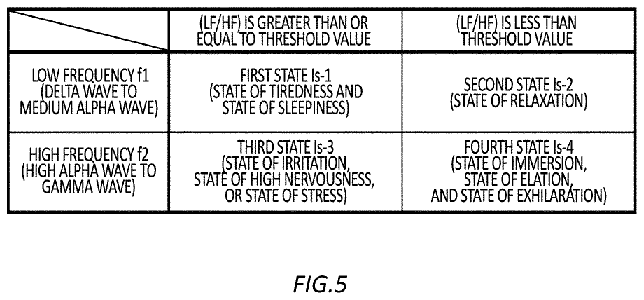

FIG. 5 is a diagram showing an example of state information Is. The state information Is may include information in relation to a plurality of states (a first state Is-1 to an nth state Is-n) of the evaluation target person 120. In the present example, the state information Is includes information in relation to four states (first state Is-1 to fourth state Is-4) of the evaluation target person 120. In FIG. 5, the brainwave of a low frequency f1 refers to at least one of the delta wave, the theta wave, the low alpha wave, or the medium alpha wave; and the brainwave of a high frequency f2 refers to at least one of the high alpha wave, the low beta wave, high beta wave, or the gamma wave.

The amplitude of the brainwave of the evaluation target person 120, which is the amplitude of the brainwave in a predetermined frequency band is set as an amplitude Af. The amplitude Af of the brainwave of the evaluation target person 120 before the first reaction R1 is set as an amplitude Af1. The amplitude Af of the brainwave of the evaluation target person 120 after the first reaction R1 is set as an amplitude Af2. The brainwave in the predetermined frequency band may be at least one of the low alpha wave, the medium alpha wave, the high alpha wave, the low beta wave, the high beta wave, the gamma wave, or the theta wave.

The reaction determination unit 20 may generate the state information Is, based on a change from a ratio of the amplitude Af1 to the total amplitude As, to a ratio of the amplitude Af2 to the total amplitude As; and the ratio of the LF to the HF (LF/HF). The state information Is may be the state information Is in relation to one state of the plurality of states of the evaluation target person 120 (any of the first state Is-1 to the nth state Is-n).

In the present example, the first state Is-1 is the state of the evaluation target person 120 when a ratio of the amplitude Af2 to the total amplitude As in the brainwave of the low frequency f1, is greater than a ratio of the amplitude Af1 to the total amplitude As, and the ratio of LF to HF (LF/HF) after the first reaction R1 is higher than or equal to a threshold value. When the evaluation target person 120 is in the first state Is-1, it can be inferred that a state of tiredness and a state of sleepiness of the evaluation target person 120 is increasing. When the evaluation target person 120 is in the first state Is-1, the reaction determination unit 20 (refer to FIG. 3) may generate the state information Is indicating that the state of tiredness and the state of sleepiness of the evaluation target person 120 is increasing. The reaction determination unit 20 may determine the second reaction R2 based on the state information Is in relation to the first state Is-1.

In the present example, the second state Is-2 is the state of the evaluation target person 120 when a ratio of the amplitude Af2 to the total amplitude As in the brainwave of the low frequency f1, is greater than a ratio of the amplitude Af1 to the total amplitude As, and the ratio of LF to HF (LF/HF) after the first reaction R1 is less than a threshold value. When the evaluation target person 120 is in the second state Is-2, it can be inferred that the state of relaxation of the evaluation target person 120 is increasing. When the evaluation target person 120 is in the second state Is-2, the reaction determination unit 20 (refer to FIG. 3) may generate the state information Is indicating that a degree of calm of the evaluation target person 120 is increasing. The reaction determination unit 20 may determine the second reaction R2, based on the state information Is in relation to the second state Is-2. An utterance “Let's go now” by the communication device 100 in FIG. 1 is an example of the second reaction R2 based on the state of relaxation of the evaluation target person 120.

In the present example, the third state Is-3 is the state of the evaluation target person 120 when a ratio of the amplitude Af2 to the total amplitude As in the brainwave of the high frequency f2, is greater than a ratio of the brainwave Af1 to the total amplitude As, and the ratio of LF to HF (LF/HF) after the first reaction R1 is higher than or equal to a threshold value. When the evaluation target person 120 is in the third state Is-3, it can be inferred that the state of irritation, the state of high nervousness, or the state of stress of the evaluation target person 120 is increasing.

When the evaluation target person 120 is in the third state Is-3, the reaction determination unit 20 (refer to FIG. 2) may generate the state information Is indicating that the state of irritation, the state of high nervousness, or the state of stress of the evaluation target person 120 is increasing. The reaction determination unit 20 (refer to FIG. 3) may determine the second reaction R2, based on the state information Is in relation to the third state Is-3. An utterance of the communication device 100 “Would you like to go for a walk? You will be refreshed” in FIG. 1 is an example of the second reaction R2 based on the state of stress of the evaluation target person 120.

In the present example, the fourth state Is-4 is the state of the evaluation target person 120 when a ratio of the amplitude Af2 to the total amplitude As in the brainwave of the high frequency f2, is greater than a ratio of the brainwave Af1 to the total amplitude As, and the ratio of LF to HF (LF/HF) after the first reaction R1 is less than a threshold value. When the evaluation target person 120 is in the fourth state Is-4, it can be inferred that the state of immersion, a state of elation, and a state of exhilaration of the evaluation target person 120 is increasing. When the evaluation target person 120 is in the fourth state Is-4, the reaction determination unit 20 (refer to FIG. 3) may generate the state information indicating that the state of immersion of the evaluation target person 120 is increasing. The reaction determination unit 20 may determine the second reaction R2, based on the state information Is in relation to the fourth state Is-4.

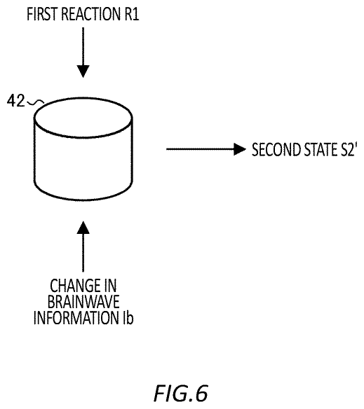

FIG. 6 is a diagram showing an example of a state inference model 42. The state learning unit 40 (refer to FIG. 3) performs machine learning on a relationship between the first reaction R1, and the change from the brainwave information Ib1 to the brainwave information Ib2. The state learning unit 40 generates the state inference model 42 by performing the machine learning on the relationship between the first reaction R1, and the change from the brainwave information Ib1 to the brainwave information Ib2.

The state inference model 42 infers the second state S2 of the evaluation target person 120, based on the brainwave information Ib1 and the first reaction R1. The inferred second state S2 is set as a second state S2′. The second state S2′ inferred based on the brainwave information Ib1 and the first reaction R1, is the state S of the evaluation target person 120 based on brainwave information Ib2′. The state inference model 42 performs the machine learning on the relationship between the first reaction R1, and the change from the brainwave information Ib1 to the brainwave information Ib2, and thus the second state S2′ can be inferred based on the relationship between the brainwave information Ib1 and the first reaction R1. Accordingly, it is possible for the evaluation target person 120 to infer the second state S2′ of the evaluation target person 120 themself after the first reaction R1. The state inference model 42 may be stored in the storage unit 50 (refer to FIG. 3).

The state learning unit 40 (refer to FIG. 3) may perform the machine learning on the relationship between the first reaction R1, and the change from the brainwave information Ib1 to the brainwave information Ib2, for a plurality of evaluation target persons 120. The state learning unit 40 may generate the state inference model 42 by performing the machine learning on the relationship between the first reaction R1, and the change from the brainwave information Ib1 to the brainwave information Ib2, for the plurality of evaluation target persons 120.

The communication device 100 may include the recognition unit 12 (refer to FIG. 3) which recognizes the evaluation target person 120. The recognition unit 12 may be included in the information acquisition unit 10. The recognition unit 12 is, for example, an image sensor, a microphone, or the like. When the recognition unit 12 is an image sensor, the recognition unit 12 identifies one evaluation target person 120 and another evaluation target person 120 by a captured image. When the recognition unit 12 is a microphone, the recognition unit 12 identifies one evaluation target person 120 and another evaluation target person 120, by the frequency of a voice of the one evaluation target person 120 and the frequency of a voice of the other evaluation target person 120.

The state learning unit 40 may perform the machine learning on the relationship between the first reaction R1, and the change from the brainwave information Ib1 to the brainwave information Ib2, for the evaluation target person 120 recognized by the recognition unit 12. The state learning unit 40 may generate the state inference model 42 for each evaluation target person 120.

The information acquisition unit 10 (refer to FIG. 3) may acquire the brainwave information Ib2. The reaction determination unit 20 (refer to FIG. 3) may determine the second reaction R2, based on the brainwave information Ib2 and the second state S2′. As described above, the second state S2′ is inferred by the state inference model 42. The state inference model 42 performs the machine learning on the relationship between the first reaction R1, and the change from the brainwave information Ib1 to the brainwave information Ib2. Therefore, by the second reaction R2 being determined based on the brainwave information Ib2 and the second state S2′, the second reaction R2 can be a more appropriate reaction for the evaluation target person 120.

A difference between the second state S2 based on the brainwave information Ib2, and the second state S2′ is set as a difference d. The difference d may be a difference between a magnitude of a specific frequency component in any of the alpha wave, the beta wave, the theta wave, the delta wave, and the gamma wave in the brainwave information Ib2, and a magnitude of the specific frequency component in the brainwave information Ib2′; or may be a difference between a value obtained by dividing the magnitude of one component among the alpha wave, the beta wave, the theta wave, the delta wave, and the gamma wave in the brainwave information Ib2, by the magnitude of another component, and a value obtained by the dividing in the brainwave information Ib2′.

The reaction determination unit 20 (refer to FIG. 3) may determine a predetermined reaction as the second reaction R2, in a case where the difference d exceeds a predetermined threshold value dth. The case where the difference d exceeds the threshold value dth is a case where the second state S2 of the evaluation target person 120 deviates from the inferred second state S2′. The predetermined reaction is, for example, an apology, a confirmation, or the like. When the second state S2 and the second state S2′ are deviated from each other, there is a high probability that the second reaction R2 determined based on the second state S2′, is an inappropriate reaction for the evaluation target person 120. Therefore, the control unit 90 (refer to FIG. 3) controls the communication device 100 to show a predetermined reaction. For example, when the state inference model 42 infers the second state S2′ indicating the state of relaxation, even though the second state S2 is the state of stress of the evaluation target person 120, the reaction determination unit 20 may determine, as the second reaction R2, an utterance of an apology such as “I'm sorry for not understanding your feelings”, or an utterance of a confirmation such as “You are still anxious”.

The reaction determination unit 20 (refer to FIG. 3) may determine the first reaction R1, based on the brainwave information Ib1 and a time at which the first reaction R1 is determined. The potential first state S1 (refer to FIG. 1 and FIG. 2) of the evaluation target person 120 may vary depending on the time at which the first reaction R1 is determined. For example, in a case where the time at which the first reaction is determined is early in the morning, the evaluation target person 120 may be in a state of tension; in a case where the time is after lunch, the evaluation target person 120 may be in the state of relaxation; and in a case where the time is in the evening, the evaluation target person 120 may be in the state of stress; and in a case where the time is late at night, the evaluation target person 120 may be in the state of sleepiness. Therefore, by the first reaction R1 being determined based on the brainwave information Ib1 and the time at which the first reaction R1 is determined, the first reaction R1 can be a more appropriate reaction for the evaluation target person 120.

The state learning unit 40 (refer to FIG. 3) may perform, for each time period, the machine learning on the relationship between the first reaction R1, and the change from the brainwave information Ib1 to the brainwave information Ib2. As described above, the potential first state S1 of the evaluation target person 120 may vary depending on the time. Therefore, by the state learning unit 40 performing, for each time period, the machine learning on the relationship between the first reaction R1, and the change from the brainwave information Ib1 to the brainwave information Ib2, the state inference model 42 can more accurately infer the second state S2′ of the evaluation target person 120. Therefore, the reaction determination unit 20 (refer to FIG. 3) can determine the more appropriate second reaction R2 for each time period, based on the brainwave information Ib2 and the second state S2′.

The reaction determination unit 20 (refer to FIG. 3) may determine the first reaction R1, based on the brainwave information Ib1 and an environment around the evaluation target person 120. The environment around the evaluation target person 120 may be acquired by the information acquisition unit 10 (refer to FIG. 3). When the recognition unit 12 (refer to FIG. 3) is the image sensor, the environment around the evaluation target person 120 may be acquired by the image sensor.

The potential first state S1 (refer to FIG. 1 and FIG. 2) of the evaluation target person 120 may vary depending on the environment around the evaluation target person 120. For example, when the weather around the evaluation target person 120 is sunny, the evaluation target person 120 may be in a state of calm, and when the weather is cloudy, the evaluation target person 120 may be in the state of anxiety. Therefore, by the first reaction R1 being determined based on the brainwave information Ib1 and the environment around the evaluation target person 120, the first reaction R1 can be a more appropriate reaction for the evaluation target person 120.

The control unit 90 may change the living body 110 which is presented to the reaction presentation unit 30 according to the evaluation target person 120. The changing the living body may refer to changing the virtual person, or may refer to changing the virtual animal such as a dog or a cat. When the control unit 90 changes the virtual person, the control unit 90 may change a gender or an age group of the virtual person.

The recognition unit 12 (refer to FIG. 3) may recognize the gender or the age group of the evaluation target person 120. In the example of FIG. 1 and FIG. 2, a young woman is presented as the virtual person in the reaction presentation unit 30. In the example of FIG. 1 and FIG. 2, when the evaluation target person 120 is female, the control unit 90 may change the virtual person who is presented to the reaction presentation unit 30, to a male virtual person.

When the second state S2 of the evaluation target person 120 is a predetermined state of the evaluation target person 120, the reaction determination unit 20 (refer to FIG. 3) may determine a predetermined reaction as the second reaction R2. The reaction determination unit 20 may determine whether the evaluation target person 120 is in the predetermined state, based on the magnitude of the frequency component of any of the alpha wave, the beta wave, the theta wave, the delta wave, and the gamma wave in the brainwave information Ib. The predetermined state is a state in which it is difficult to change the second state S2 of the evaluation target person 120 by the second reaction R2. The predetermined state is, for example, a state of deep irritation, a state of deep disappointment, a state of deep ecstasy, or the like. The case where the evaluation target person 120 is in the predetermined state may refer to a case where the first reaction R1 could not have been a reaction that corresponds to the potential first state S1 of the evaluation target person 120. In such a case, the reaction determination unit 20 may determine the predetermined reaction as the second reaction R2. When the predetermined state is the state of deep irritation, the predetermined reaction is, for example, an utterance that confirms the second state S2 of the evaluation target person 120, such as “I'm sorry that I can't find a better word”.

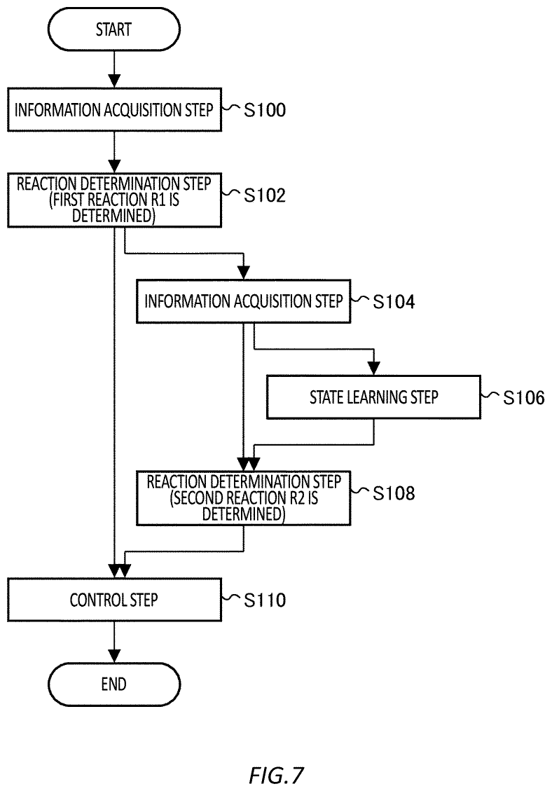

FIG. 7 is a flowchart showing an example of a communication method according to an embodiment of the present invention. The communication method according to an embodiment of the present invention will be described by using the communication device 100 shown in FIG. 3 as an example.

The communication method includes an information acquisition step S100, a reaction determination step S102, and a control step S110. The communication method may include an information acquisition step S104, a state learning step S106, and a reaction determination step S108.

The information acquisition step S100 is a step in which the information acquisition unit 10 acquires the brainwave information Ib1 of the evaluation target person 120. The reaction determination step S102 is a step in which the reaction determination unit 20 determines the first reaction R1 of the communication device 100, based on the brainwave information Ib1. The control step S110 is a step in which the control unit 90 controls the communication device 100 according to the first reaction R1 determined in the reaction determination step S102.

The reaction determination step S102 may be a step in which the reaction determination unit 20 determines the content of the next utterance of the communication device 100, based on the brainwave information Ib1. The control step S110 may be a step of controlling the communication device 100 according to the content of the utterance determined in the reaction determination step S102.

The information acquisition step S100 may be a step in which the information acquisition unit 10 further acquires the biological information Ig of the evaluation target person 120. The information acquisition step S100 may be a step in which the information acquisition unit 10 further acquires the biological information Ig of the evaluation target person 120 before the first reaction R1. The reaction determination step S102 may be a step in which the reaction determination unit 20 determines the first reaction R1, based on the brainwave information Ib and the biological information Ig.

The information acquisition step S104 may be a step in which the information acquisition unit 10 acquires the brainwave information Ib2 after the first reaction R1 of the communication device 100. The information acquisition step S104 may be a step in which the information acquisition unit 10 further acquires the biological information Ig of the evaluation target person 120 after the first reaction R1 of the communication device 100. The reaction determination step S108 may be a step in which the reaction determination unit 20 determines the second reaction R2 of the communication device 100, based on the brainwave information Ib2 and the biological information Ig. The control step S110 may be a step in which the control unit 90 controls the communication device 100 according to the second reaction R2.

The information acquisition step S104 is a step in which the information acquisition unit 10 acquires the change in the brainwave information Ib from before to after the first reaction R1 of the communication device 100. The information acquisition step S104 may be a step in which the information acquisition unit 10 acquires the brainwave information Ib2 after the first reaction R1. The information acquisition step S104 may be a step in which the information acquisition unit 10 acquires the change from the brainwave information Ib1 to the brainwave information Ib2.

The reaction determination step S108 may be a step in which the reaction determination unit 20 generates the state information Is, based on the change in the brainwave information Ib acquired in the information acquisition step S104, and the biological information Ig, and determines the second reaction R2 based on the generated state information Is. The reaction determination step S108 may be a step of determining the second reaction R2 of the communication device 100.

The reaction determination step S108 may be a step in which the reaction determination unit 20 generates the state information Is, based on a change from a ratio of an amplitude of a brainwave in a predetermined frequency band to the total amplitude As in the brainwave information Ib before the first reaction R1, to a ratio of an amplitude of a brainwave in the frequency band to the total amplitude As in the brainwave information Ib after the first reaction R1; and the ratio of the LF to the HF (LF/HF) in the heart rate of the evaluation target person 120.

The reaction determination step S108 may be a step in which the reaction determination unit 20 generates the state information Is, based on a change from a ratio of an amplitude of a brainwave in a predetermined frequency band to the total amplitude As in the brainwave information Ib before the first reaction R1, to a ratio of an amplitude of a brainwave in the frequency band to the total amplitude As in the brainwave information Ib after the first reaction R1; and a magnitude relationship between the ratio of the LF to the HF (LF/HF) after the first reaction R1, and a predetermined threshold value of the ratio of the LF to the HF.

The state information Is may include information in relation to the plurality of states of the evaluation target person 120. The reaction determination step S108 may be a step in which the reaction determination unit 20 generates the state information Is in relation to one state among the plurality of states of the evaluation target person 120, based on a change from a ratio of an amplitude of a brainwave in a predetermined frequency band to the total amplitude As in the brainwave information Ib before the first reaction R1, to a ratio of an amplitude of a brainwave in the frequency band to the total amplitude As in the brainwave information Ib after the first reaction R1; and the ratio of the LF to the HF (LF/HF) in the heart rate of the evaluation target person 120.

The reaction determination step S102 may be a step in which when the second state S2 based on the brainwave information Ib2 of the evaluation target person 120 is a predetermined state of the evaluation target person 120, the reaction determination unit 20 determines a predetermined reaction as the second reaction R2.

The state learning step S106 is a step of generating a state inference model that infers the state S2′ of the evaluation target person 120 after the first reaction R1, by performing the machine learning on the relationship between the first reaction R1 and the change in brainwave information Ib, based on the brainwave information Ib1 before the first reaction R1 and the first reaction R1.

The information acquisition step S104 may be a step in which the information acquisition unit 10 acquires the brainwave information Ib2 after the first reaction R1 of the communication device 100. The reaction determination step S108 may be a step in which the reaction determination unit 20 determines the second reaction R2 of the communication device 100, based on the second state S2 of the evaluation target person 120 based on the brainwave information Ib2 acquired in the information acquisition step S104, and the second state S2′ of the evaluation target person 120 inferred in the state learning step S106. The reaction determination step S108 may be a step in which when the difference d between the second state S2 and the second state S2′ exceeds a predetermined threshold value, the reaction determination unit 20 determines a predetermined reaction as the second reaction R2.

The reaction determination step S102 may be a step in which the reaction determination unit 20 determines the first reaction R1, based on the time at which the first reaction R1 is determined, or the environment around the evaluation target person 120. The control step S110 may be a step in which the control unit 90 changes the living body 110 which is presented to the reaction presentation unit 30 according to the evaluation target person 120.

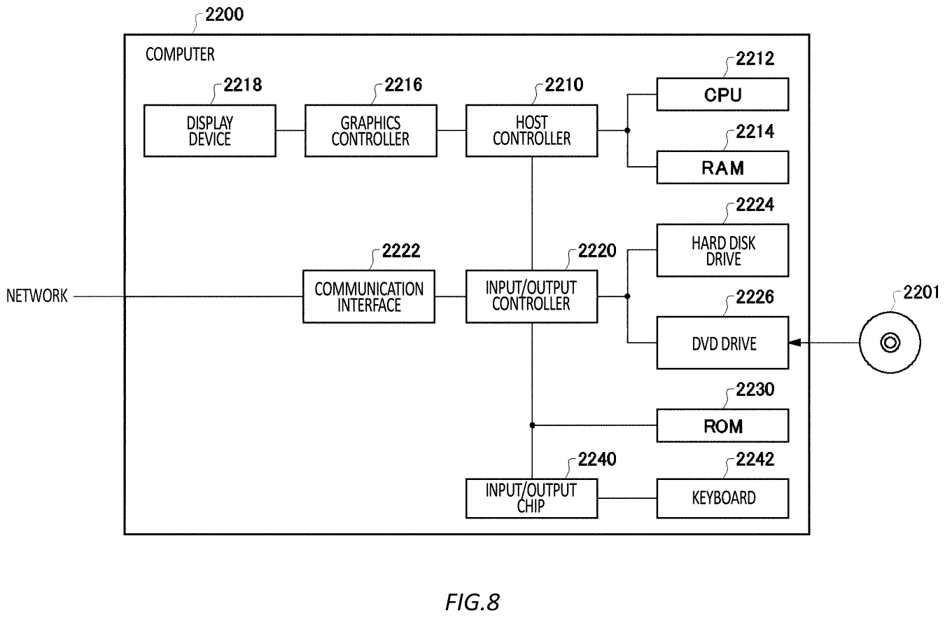

FIG. 8 is a diagram showing an example of a computer 2200 in which a communication device 100 according to an embodiment of the present invention may be implemented in whole or in part. A program installed on the computer 2200 can cause the computer 2200 to perform operations associated with the communication device 100 according to embodiments of the present invention, or to function as one or more sections of the communication device 100, or can cause the computer 2200 to execute the operations or the one or more sections thereof, and/or can cause the computer 2200 to execute each step (refer to FIG. 7) according to the method of the present invention. Such a program may be executed by a CPU 2212 to cause a computer 2200 to execute certain operations associated with some or all of the blocks of flowcharts (refer to FIG. 7) and block diagrams (refer to FIG. 3) described herein.

The computer 2200 according to an embodiment of the present invention includes a CPU 2212, a RAM 2214, a graphics controller 2216, and a display device 2218. The CPU 2212, the RAM 2214, the graphics controller 2216, and the display device 2218 are mutually connected by a host controller 2210. The computer 2200 further includes input/output unit such as a communication interface 2222, a hard disk drive 2224, a DVD-ROM drive 2226, and an IC card drive. The communication interface 2222, the hard disk drive 2224, the DVD-ROM drive 2226, and the IC card drive, and the like are connected to the host controller 2210 via an input/output controller 2220. The computer further includes legacy input/output units such as a ROM 2230 and a keyboard 2242. The ROM 2230, the keyboard 2242, and the like are connected to the input/output controller 2220 via an input/output chip 2240.

The CPU 2212 operates according to programs stored in the ROM 2230 and the RAM 2214, thereby controlling each unit. The graphics controller 2216 acquires image data generated by the CPU 2212 on a frame buffer or the like provided in the RAM 2214 or in the RAM 2214 itself to cause the image data to be displayed on the display device 2218.

The communication interface 2222 communicates with other electronic devices via a network. The hard disk drive 2224 stores programs and data used by the CPU 2212 in the computer 2200. The DVD-ROM drive 2226 reads the programs or the data from a DVD-ROM 2201, and provides the read programs or the data to the hard disk drive 2224 via the RAM 2214. The IC card drive reads the programs and the data from an IC card, or writes the programs and the data to the IC card.

The ROM 2230 stores a boot program or the like executed by the computer 2200 at the time of activation, or a program depending on the hardware of the computer 2200. The input/output chip 2240 may connect various input/output units via a parallel port, a serial port, a keyboard port, a mouse port, or the like to the input/output controller 2220.

The program is provided by a computer-readable medium such as the DVD-ROM 2201 or the IC card. The program is read from the computer-readable medium, installed in the hard disk drive 2224, the RAM 2214, or the ROM 2230 which are also examples of the computer-readable medium, and executed by the CPU 2212. The information processing written in these programs is read by the computer 2200 and provides cooperation between the programs and the above-described various types of hardware resources. An apparatus or method may be constituted by realizing the operation or processing of information in accordance with the usage of the computer 2200.

For example, when a communication is executed between the computer 2200 and an external device, the CPU 2212 may execute a communication program loaded onto the RAM 2214 to instruct the communication interface 2222 to process the communication, based on the processing written in the communication program. The communication interface 2222, under control of the CPU 2212, reads transmission data stored on a transmission buffering region provided in a recording medium such as the RAM 2214, the hard disk drive 2224, the DVD-ROM 2201, or the IC card, and transmits the read transmission data to a network or writes reception data received from a network to a reception buffering region or the like provided on the recording medium.

The CPU 2212 may cause all or a necessary portion of a file or a database to be read into the RAM 2214, the file or the database having been stored in an external recording medium such as the hard disk drive 2224, the DVD-ROM drive 2226 (DVD-ROM 2201), the IC card, or the like. The CPU 2212 may execute various types of processing on the data on the RAM 2214. The CPU 2212 may then write back the processed data to the external recording medium.

Various types of information, such as various types of programs, data, tables, and databases, may be stored in the recording medium to undergo information processing. The CPU 2212 may execute various types of processing on the data read from the RAM 2214, which includes various types of operations, information processing, condition judging, conditional branch, unconditional branch, search or replacement of information, or the like, as described throughout the present disclosure and designated by an instruction sequence of programs. The CPU 2212 may write the result back to the RAM 2214.

The CPU 2212 may search for information in a file, a database, or the like in the recording medium. For example, when a plurality of entries, each having an attribute value of a first attribute associated with an attribute value of a second attribute, are stored in the recording medium, the CPU 2212 may search for an entry matching the condition whose attribute value of the first attribute is designated, from among the plurality of entries, read the attribute value of the second attribute stored in the entry, and read a second attribute value to acquire the attribute value of the second attribute associated with the first attribute satisfying the predetermined condition.

The program or software modules described above may be stored on the computer 2200 or in the computer-readable medium of the computer 2200. A recording medium such as a hard disk or a RAM provided in a server system connected to a dedicated communication network or the Internet can be used as the computer-readable medium. The program may be provided to the computer 2200 by the recording medium.

While the present invention has been described with the embodiments, the technical scope of the present invention is not limited to the above embodiments. It is apparent to persons skilled in the art that various alterations and improvements can be added to the above-described embodiments. It is also apparent from the description of the claims that embodiments added with such alterations or improvements can be included in the technical scope of the present invention.

The operations, procedures, steps, and stages of each process performed by an apparatus, system, program, and method illustrated in the claims, embodiments, or diagrams can be performed in any order as long as the order is not indicated by “prior to,” “before,” or the like and as long as the outputted from a previous process is not used in a later process. Even if the operation flow is described by using phrases such as “first” or “next” in the scope of the claims, specification, or drawings, it does not necessarily mean that the process must be performed in this order.

### EXPLANATION OF REFERENCES

10: information acquisition unit; 12: recognition unit; 14: electroencephalograph; 20: reaction determination unit; 30: reaction presentation unit; 40: state learning unit; 42: state inference model; 50: storage unit; 90: control unit; 100: communication device; 110: living body; 120: evaluation target person; 2200: computer; 2201: DVD-ROM; 2210: host controller; 2212: CPU; 2214: RAM; 2216: graphics controller; 2218: display device; 2220: input/output controller; 2222: communication interface; 2224: hard disk drive; 2226: DVD-ROM drive; 2230: ROM; 2240: input/output chip; 2242: keyboard.

What is claimed is:

1. A communication device in which a reaction changes according to a state of an evaluation target person, the communication device comprising:
an information acquisition unit which acquires brainwave information of the evaluation target person;
a reaction determination unit which determines a first reaction of the communication device, based on the brainwave information; and
a control unit which controls the communication device according to the first reaction determined by the reaction determination unit.

2. The communication device according to claim 1, wherein
the reaction determination unit determines a content of an utterance of the communication device, based on the brainwave information, and
the control unit controls the communication device according to the content of the utterance determined by the reaction determination unit.

3. The communication device according to claim 1, wherein
the information acquisition unit further acquires biological information of the evaluation target person, and
the reaction determination unit determines the first reaction, based on the brainwave information and the biological information.

4. The communication device according to claim 2, wherein
the information acquisition unit further acquires biological information of the evaluation target person, and
the reaction determination unit determines the first reaction, based on the brainwave information and the biological information.

5. The communication device according to claim 3, wherein
the information acquisition unit further acquires the biological information after the first reaction,
the reaction determination unit determines a second reaction of the communication device, based on the brainwave information, and the biological information after the first reaction, and
the control unit controls the communication device according to the second reaction.

6. The communication device according to claim 5, wherein
the information acquisition unit acquires a change in the brainwave information from before to after the first reaction of the communication device, and
the reaction determination unit generates state information indicating a state of the evaluation target person, based on the change in the brainwave information, and the biological information, and determines the second reaction based on the generated state information.

7. The communication device according to claim 6, wherein
the reaction determination unit generates the state information, based on a change from a ratio of an amplitude of a brainwave in a predetermined frequency band to a total amplitude in the brainwave information before the first reaction, to a ratio of an amplitude of a brainwave in the frequency band to the total amplitude in the brainwave information after the first reaction; and a ratio of a magnitude of a first power spectrum to a magnitude of a second power spectrum in a heart rate of the evaluation target person,
the total amplitude is a sum of amplitudes of an alpha wave, a beta wave, a theta wave, a gamma wave, and a delta wave, and
a frequency band of the second power spectrum is a band in which a frequency is higher than that in a frequency band of the first power spectrum.

8. The communication device according to claim 7, wherein the reaction determination unit generates the state information based on the change; and a magnitude relationship between the ratio of the magnitude of the first power spectrum to the magnitude of the second power spectrum after the first reaction, and a predetermined threshold value of the ratio of the magnitude of the first power spectrum to the magnitude of the second power spectrum.

9. The communication device according to claim 7, wherein
the state information includes information in relation to a plurality of states of the evaluation target person, and
the reaction determination unit generates the state information in relation to one state among the plurality of states, based on the change; and the ratio of the magnitude of the first power spectrum to the magnitude of the second power spectrum.

10. The communication device according to claim 9, wherein the brainwave in the frequency band is at least one of the delta wave, the theta wave, a low alpha wave, or a medium alpha wave.

11. The communication device according to claim 9, wherein the brainwave in the frequency band is at least one of a high alpha wave, a low beta wave, a high beta wave, or the gamma wave.

12. The communication device according to claim 5, wherein when a state that is based on the brainwave information of the evaluation target person and that is a state after the first reaction is a predetermined state, the reaction determination unit determines a predetermined reaction as the second reaction.

13. The communication device according to claim 6, further comprising: a state learning unit which generates a state inference model that infers the state of the evaluation target person after the first reaction, by performing machine learning on a relationship between the first reaction and the change in the brainwave information, based on the brainwave information before the first reaction and the first reaction.

14. The communication device according to claim 13, wherein
the information acquisition unit acquires the brainwave information after the first reaction of the communication device, and
the reaction determination unit determines the second reaction of the communication device, based on the brainwave information after the first reaction, and the state of the evaluation target person inferred by the state inference model.

15. The communication device according to claim 13, wherein when a difference between the state of the evaluation target person based on the brainwave information after the first reaction, and the state of the evaluation target person inferred by the state inference model, exceeds a predetermined threshold value, the reaction determination unit determines a predetermined reaction as the second reaction.

16. The communication device according to claim 1, wherein the reaction determination unit determines the first reaction, based on a time at which the first reaction is determined, or an environment around the evaluation target person.

17. The communication device according to claim 2, wherein the reaction determination unit determines the first reaction, based on a time at which the first reaction is determined, or an environment around the evaluation target person.

18. The communication device according to claim 1, wherein the control unit changes a living body which is presented to a reaction presentation unit according to the evaluation target person.

19. A communication method in which a reaction changes according to a state of an evaluation target person, the communication method comprising:
acquiring, by an information acquisition unit, brainwave information of the evaluation target person;
determining, by a reaction determination unit, a first reaction of a communication device, based on the brainwave information; and
controlling, by a control unit, the communication device according to the first reaction determined in the determining the reaction.

20. A computer-readable medium having recorded thereon a communication program that, when executed by a computer, causes the computer to perform operations comprising:
acquiring brainwave information of an evaluation target person;
determining a first reaction of a communication device, based on the brainwave information; and
controlling the communication device according to the first reaction determined in the determining the reaction.
